(19) United States
(12) Patent Application Publication
(10) Pub. No.: US 2011/0144385 A1
(43) Pub. Date: Jun. 16, 2011

(54) Method For The Production Of Aryl Polyglycol Carboxylic Acids By Means Of A Direct Oxidation Process

(75) Inventors: Oliver Franke, Muenchen (DE); Achim Stankowiak, Altoetting (DE); Rainer Kupfer, Hattersheim (DE); Ulf Pruesse, Braunschweig (DE); Nadine Decker, Erkner (DE); Klaus-Dieter Vorlop, Braunschweig (DE)

(73) Assignee: CLARIANT FINANCE (BVI) LIMITED, Tortola (VG)

(21) Appl. No.: 13/057,863

(22) PCT Filed: Jul. 15, 2009
(86) PCT No.: PCT/EP2009/005134

§ 371 Date: Feb. 7, 2011

(30) Foreign Application Priority Data
Aug. 8, 2008 (DE) 10 2008 037 065.7

(51) Int. Cl. C07C 51/16

(52) U.S. Cl. 562/421

(57) ABSTRACT

The invention relates to a method for producing compounds of formula (I) in which R¹ represents an aromatic group containing 6 to 200 carbon atoms, R² represents hydrogen, a linear or branched alkyl group containing 1 to 22 carbon atoms, a monounsaturated or polyunsaturated linear or branched alkenyl group containing 2 to 22 carbon atoms, or an aryl group containing 6 to 12 carbon atoms, X represents an alkylene group containing 2 to 4 carbon atoms, n represents a number between 0 and 100, and B represents a cation or hydrogen, and/or the corresponding protonated carboxylic acids, by oxidizing one or more compounds of formula (II) in which R¹, R², X, and n have the meaning indicated above, with oxygen or oxygen-containing gases in the presence of a gold-containing catalyst and at least one alkaline compound.

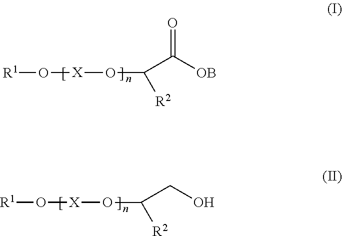

Aryl polyglycol carboxylic acids (ether carboxylic acids), i.e. organic carboxylic acids, which, besides the carboxyl function, carry one or more ether bridges, or alkali metal or amine salts thereof, are known as mild detergents with high lime soap dispersing power. They are used both in detergent and cosmetics formulations, and also in technical applications, such as, for example, metal working fluids and cooling lubricants

According to the prior art, ether carboxylic acids are synthesized either by alkylation of aryl polyglycols with chloroacetic acid derivatives (Williamson ether synthesis) or from the same starting materials by oxidation with various reagents (atmospheric oxygen, hypochlorite, chlorite) with catalysis with various catalysts. The Williamson ether synthesis is the industrially most common method for producing ether carboxylic acid, primarily on account of the cost-benefit relationship, but products produced by this method still have serious shortcomings in relation to the handleability for the user, such as, for example, solubility behavior, aggregate state at low temperatures and storage stability.

These shortcomings are essentially to be attributed to secondary constituents caused by the method. Thus, despite using excesses of the corresponding chloroacetic acid derivative, only conversions of ca. 70-85% are achieved, meaning that residual amounts of oxethylate and fatty alcohol on which the oxethylate is based remain in the end product. Furthermore, as a result of the excess of the chloroacetic acid derivative to be used, secondary products are formed, such as, for example, glycolic acid, diglycolic acid and derivatives thereof, which are a significant cause of the ageing of the products and can in some circumstances cause problems with the solubility behavior.

A further disadvantage of the Williamson synthesis is the high contamination of the reaction products by sodium chloride, which in aqueous solutions is a significant cause of pitting corrosion. Moreover, the formed sodium chloride enters the reaction wastewater, where it constitutes a problem for biological sewage plants, since sodium chloride can adversely affect the cleaning efficiency of such plants.

The direct oxidation of alcohol oxethylates to ether carboxylic acids takes place with the help of platinum catalysts, as described e.g. in U.S. Pat. No. 3,342,858. Platinum can be used both as suspension, or else be applied to a support material such as carbon. The oxidation is carried out in alkaline solution at a temperature of from 20 to 75° C. and a maximum pressure of 3 bar. Disadvantages of this method are the very dilute solutions (3 to 12% strength aqueous solutions), the sometimes long reaction times of up to 24 hours and the associated low space-time yield. The low selectivities are likewise disadvantageous with the platinum catalysts used; the yields are only ca. 68 to 89% following work-up by distillation.

Surprisingly, it has now been found that ether carboxylic acids and salts thereof are also accessible in high yield through direct oxidation of aryl polyglycols with atmospheric oxygen or pure oxygen by means of gold-containing catalysts.

The present invention therefore provides a method for producing compounds of the formula (I)

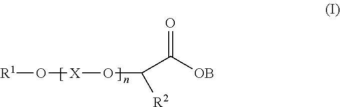

R¹ is an aromatic group having 6 to 200 carbon atoms,
R² is hydrogen, a linear or branched alkyl radical having 1 to 22 carbon atoms, a mono- or polyunsaturated linear or branched alkenyl radical having 2 to 22 carbon atoms, or an aryl radical having 6 to 12 carbon atoms,
X is an alkylene radical having 2 to 4 carbon atoms,
n is a number between 0 and 100,
B is a cation or hydrogen,
   and/or of the corresponding protonated carboxylic acids by oxidizing one or more compounds of the formula (II)

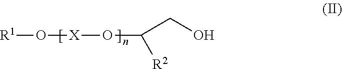

in which R¹, R², X and n have the meaning given above, with oxygen or gases containing oxygen in the presence of a gold-containing catalyst and at least one alkaline compound.

R¹ is preferably an aromatic group having 6 to 24 carbon atoms. Particularly preferably, R¹ is a pure hydrocarbon group.

The aromatic systems which are present in R¹ can be substituted with alkyl or alkenyl groups which comprise 1-200, preferably 2-20, in particular 4-16, such as, for example, 6-12, carbon atoms.

In a particularly preferred embodiment, R¹ is a phenyl group which is substituted with alkyl or alkenyl groups which comprise 1-200, preferably 2-20, in particular 4-16, such as, for example, 6-12, carbon atoms. These are preferably n-, iso- and tert-butyl radicals, n- and isopentyl radicals, n- and isohexyl radicals, n- and isooctyl radicals, n- and isononyl radicals, n- and isodecyl radicals, n- and isododecyl radicals, tetradecyl radicals, hexadecyl radicals, octadecyl radicals, tripropenyl radicals, tetrapropenyl radicals, poly(propenyl) radicals and poly(isobutenyl) radicals.

Of suitability according to the invention are in particular those aromatic systems R¹ which are derived from alkylphenols having one or two alkyl radicals in the ortho and/or para position relative to the OH group. Particularly preferred starting materials are alkylphenols which carry on the aromatic at least two hydrogen atoms capable of condensation with aldehydes, and in particular monoalkylated phenols. Particular preference is given to aromatic systems R¹ with an alkyl or alkenyl group which comprises 1-200, preferably 2-20, in particular 4-16, such as, for example, 6-12, carbon atoms, in the para position relative to the phenolic OH group.

In a further preferred embodiment, aromatic systems R¹ with different alkyl radicals are used, for example butyl radicals on the one hand, and octyl, nonyl and/or dodecyl radicals in the molar ratio of 1:10 to 10:1 on the other hand.

By way of example, R¹ is phenyl, tributylphenyl, tristyrylphenyl, nonylphenyl, cumyl or octylphenyl radicals.

Preferably, R² is hydrogen or a C₁ to C₄-alkyl radical.

The polyglycol chain (X—O) of the starting compound (II) may be a pure or mixed alkoxy chain with random or blockwise distribution of (X—O) groups.

As alkaline compounds, carbonates, hydroxides or oxides can be used in the method according to the invention. Preferably, the hydroxides are BOH.

The counterions B are preferably alkali metal cations selected from cations of the alkali metals Li, Na, K, Rb and Cs. The cations of the alkali metals are particularly preferably Na and K. As alkaline compound in the method according to the invention, the hydroxides of Li, Na, K, Rb and Cs are particularly preferred.

The gold-containing catalyst may be a pure gold catalyst or a mixed catalyst which comprises further metals of group VIII as well as gold. Preferred catalysts are gold catalysts which are additionally doped with one of the metals from group VIII. Particular preference is given to doping with platinum or palladium.

Preferably, the metals are applied to supports. Preferred supports are activated carbon or oxidic supports, preferably titanium dioxide, cerium dioxide or aluminum oxide. Such catalysts can be prepared by the known methods, such as incipient wetness (IW) or deposition precipitation (DP) as described e.g. in L. Prati, G. Martra, Gold Bull. 39 (1999) 96 and S. Biella, G. L. Castiglioni, C. Fumagalli, L. Prati, M. Rossi, Catalysis Today 72 (2002) 43-49 or L. Prati, F. Porta, Applied catalysis A: General 291 (2005) 199-203.

The supported pure gold catalysts comprise preferably 0.1 to 5% by weight of gold, based on the weight of the catalyst, which consists of support and gold.

If the catalyst comprises gold and a further metal, then this is preferably 0.1 to 5% by weight of gold and 0.1 to 3% by weight of a group VIII metal, preferably platinum or palladium. Particular preference is given to those catalysts which comprise 0.5 to 3% by weight of gold. The preferred gold/group VIII metal weight ratio, in particular gold/platinum or gold/palladium, is 70:30 to 95:5.

In a further preferred embodiment, the pure gold catalyst is a nanogold catalyst with a particle size of preferably 1 to 50 nm, particularly preferably 2 to 10 nm. Pure nanogold catalysts comprise preferably 0.1 to 5% by weight of gold, particularly preferably 0.5 to 3% by weight, of gold. If the catalyst comprises nanogold and a further metal, then this is preferably 0.1 to 5% by weight of nanogold and 0.1 to 2% by weight of a group VIII metal, preferably platinum or palladium. Particular preference is given to those catalysts which comprise 0.5 to 3% by weight of nanogold. The preferred nanogold/group VIII metal weight ratio, in particular nanogold/platinum or nanogold/palladium, is 70:30 to 95:5.

The method according to the invention is preferably carried out in water.

The oxidation reaction is carried out at a temperature of from 30 to 200° C., preferably between 80 and 150° C.

The pH during the oxidation is preferably between 8 and 13, particularly preferably between 9 and 11.

The pressure during the oxidation reaction is preferably increased compared to atmospheric pressure.

During the reaction in the alkaline medium, firstly the alkali metal salts (B=Li, Na, K, Rb, Cs) of the carboxylic acids are formed, preferably the sodium or potassium salts. To produce the free ether carboxylic acid (i.e. B=hydrogen), the resulting ether carboxylates of the formula (I) are reacted with acids. Preferred acids are hydrochloric acid and sulfuric acid.

The method according to the invention produces preferably solutions of carboxylates of the formula (I) with only still small residual content of aryl polyglycols of the formula (II) of <10% by weight, preferably <5% by weight, particularly preferably <2% by weight.

### EXAMPLES

### Example 1

1 liter of an aqueous 10% strength by weight tristyrylphenol polyethylene glycol solution (16 EO, M_{W}=1100 g/mol) is added to a 2 liter pressurized autoclave with gas-dispersion stirrer. After adding 10 g of a nanogold catalyst (0.9% by weight of gold and 0.1% by weight of platinum on cerium dioxide, particle size 4 to 8 nm), the suspension is adjusted to pH 10 with sodium hydroxide solution and heated to 120° C. After reaching the reaction temperature, the reaction solution is injected with oxygen at a pressure of 10 bar and held at this pressure by after-injection. Throughout the entire reaction time, the pH of the mixture is kept at 10 with sodium hydroxide solution by means of an autotitrator. After 4 hours, the reactor is cooled and decompressed, and the catalyst is separated off from the reaction solution by filtration. The solution exhibits a content of ca. 10% by weight of tristyrylphenol polyethylene glycol carboxylate, tristyrylphenol polyethylene glycol can no longer be detected.

### Example 2

1 liter of an aqueous 10% strength by weight nonylphenol polyethylene glycol solution (6EO, M_{W}=490 g/mol) is added to a 2 liter pressurized autoclave with gas-dispersion stirrer. After adding 10 g of a gold catalyst (0.9% by weight of gold and 0.1% by weight of platinum on titanium dioxide, particle size 4 to 8 nm), the suspension is adjusted to pH 11 with sodium hydroxide solution and heated to 110° C. After reaching the reaction temperature, the reaction solution is injected with oxygen at a pressure of 8 bar and held at this pressure by after-injection. Throughout the entire reaction time, the pH of the mixture is kept at 11 with sodium hydroxide solution by means of an autotitrator. After 2 hours, the reactor is cooled and decompressed, and the catalyst is separated off from the reaction solution by filtration. The solution exhibits a content of ca. 10% by weight of nonylphenol polyglycol carboxylate, nonylphenol ethoxylate can no longer be detected.

### Example 3

1 liter of an aqueous 10% strength by weight tri-sec-butylphenol polyethylene glycol solution (6 EO, M_{W}=530 g/mol) is added to a 2 liter pressurized autoclave with gas-dispersion stirrer. After adding 10 g of a gold catalyst (0.9% by weight of gold and 0.1% by weight of platinum on titanium dioxide, particle size 4 to 8 nm), the suspension is adjusted to pH 11 with sodium hydroxide solution and heated to 100° C. After reaching the reaction temperature, the reaction solution is injected with oxygen at a pressure of 8 bar and held at this pressure by after-injection. Throughout the entire reaction time, the pH of the mixture is kept at 11 with sodium hydroxide solution by means of an autotitrator. After 3 hours, the reactor is cooled and decompressed, and the catalyst is separated off from the reaction solution by filtration. The solution exhibits a content of ca. 10% by weight of tri-sec-butylphenol polyethylene glycol carboxylate, tri-sec-butylphenol polyethylene glycol can no longer be detected.

1. A method for producing compounds of the formula (I) 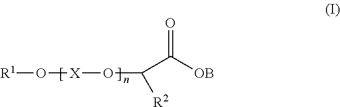
R¹ is an aromatic group having 6 to 200 carbon atoms,
R² is hydrogen, a linear or branched alkyl radical having 1 to 22 carbon atoms, a mono- or polyunsaturated linear or branched alkenyl radical having 2 to 22 carbon atoms, or an aryl radical having 6 to 12 carbon atoms,
X is an alkylene radical having 2 to 4 carbon atoms,
n is a number between 0 and 100,
B is a cation or hydrogen,
and/or of the corresponding protonated carboxylic acids by oxidizing one or more compounds of the formula (II) 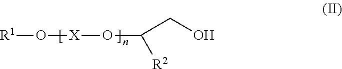
in which R¹, R², X and n have the meaning given above, with oxygen or gases containing oxygen in the presence of a gold-containing catalyst and at least one alkaline compound.

2. The method as claimed in claim 1, wherein the gold-containing catalyst is a nanogold catalyst with an average particle size of from 1 to 50 nm.

3. The method as claimed in claim 2, wherein the nanogold catalyst is applied to an oxidic support or to carbon.

4. The method as claimed in claim 3, wherein the oxidic support comprises titanium dioxide, aluminum oxide or cerium dioxide.

5. The method as claimed in one or more of claims 2 to 4, wherein the nanogold catalyst comprises 0.1 to 5% by weight of nanogold.

6. The method as claimed in one or more of claims 2 to 5, wherein the nanogold catalyst comprises 0.1 to 5% by weight of nanogold and 0.1 to 2% by weight of a group VIII metal.

7. The method as claimed in one or more of claims 1 to 6, wherein the gold-containing catalyst comprises gold and a further element of group VIII in the weight ratio Au:group VIII metal=70:30 to 95:5.

8. The method as claimed in one or more of claims 1 to 7, wherein R¹ is an aromatic group having 6 to 24 carbon atoms.

9. The method as claimed in one or more of claims 1 to 8, wherein R¹ is a hydrocarbon group.

10. The method as claimed in one or more of claims 1 to 9, wherein the aromatic systems present in R¹ are substituted with alkyl or alkenyl groups having 1 to 200 carbon atoms.

11. The method as claimed in one or more of claims 1 to 10, wherein R¹ is selected from phenyl, tributylphenyl, tristyrylphenyl, nonylphenyl or octylphenyl groups, and also from phenyl groups which are substituted with n-, iso- and tert-butyl radicals, n- and isopentyl radicals, n- and isohexyl radicals, n- and isooctyl radicals, n- and isononyl radicals, n- and isodecyl radicals, n- and isododecyl radicals, tetradecyl radicals, hexadecyl radicals, octadecyl radicals, tripropenyl radicals, tetrapropenyl radicals, poly(propenyl) radicals and poly(isobutenyl) radicals.

12. The method as claimed in one or more of claims 1 to 11, wherein R² is hydrogen or a C₁ to C₄-alkyl radical.

13. The method as claimed in one or more of claims 1 to 12, wherein B is hydrogen or a cation of the alkali metals Li, Na, K, Rb and Cs.
